(19) United States
(12) Patent Application Publication
(10) Pub. No.: US 2010/0062414 A1
(43) Pub. Date: Mar. 11, 2010

(54) SAMPLE LIQUID ANALYTICAL CHIP

(75) Inventors: Tomohiro Yamamoto, Osaka (JP); Tetsuo Yukimasa, Kanagawa (JP); Ryou Hamada, Kanagawa (JP); Fumihisa Kitawaki, Kanagawa (JP); Nobuhiko Ozaki, Kanagawa (JP)

Correspondence Address: GREENBLUM & BERNSTEIN, P.L.C., 1950 ROLAND CLARKE PLACE, RESTON, VA, 20191, US

(73) Assignee: PANASONIC CORPORATION, Osaka (JP)

(21) Appl. No.: 12/593,986

(22) PCT Filed: Apr. 4, 2008
(86) PCT No.: PCT/JP2008/000881

§ 371 Date: Sep. 30, 2009

(30) Foreign Application Priority Data
Apr. 5, 2007 (JP) 2007-099693

(51) Int. Cl. C12Q 1/00; G01N 31/22; C12M 1/34; G01N 21/00; H01R 43/00

(52) U.S. Cl. 435/4; 422/68.1; 435/287.1; 436/164; 29/825

(57) ABSTRACT

A sample liquid analytical chip that whilst being of simple structure, has a reaction region and a detection region isolated from each other and further has liquid feed control means for a sample liquid, thereby realizing speedy reliable analysis. The sample liquid analytical chip comprises a tubular sample liquid transfer channel with its both ends open, a reaction reagent layer disposed on the internal wall surface of the sample liquid transfer channel and a detection region for detecting any physical or chemical change of the sample liquid. In the sample liquid analytical chip, the sample liquid transfer channel at an area of its wall surface is provided an opening and a lid fitted to the opening. Portion of the lid forms a hinge in cooperation with the wall surface.

### TECHNICAL FIELD

The present invention relates to an analysis chip that allows a liquid sample to react with a solid reagent to analyze the liquid sample.

### BACKGROUND ART

In recent years, advanced analysis technology and testing technology have enabled various types of substances to be measured. Particularly, in the fields of clinical testing, development of measurement systems based on specific reactions such as biochemical reactions, enzyme reactions and antigen-antibody reactions has made it possible to measure substances in bodily fluids, which reflects clinical conditions. The field of clinical testing called “Point of Care Testing (POCT)” is particularly popular. In POCT, various efforts for simple and quick measurement are directed to shortening the period from when a sample is collected until the testing result is found out. That is, POCT requires measurement apparatuses that adopt simple measurement systems, that is compact and portable, and that offers good operability.

To meet this demand, various methods are proposed to measure specific types of substances in a sample in a simple manner, without diluting or stirring sample liquid. Representative methods include (1) the use of an enzyme reaction and (2) the use of antigen-antibody reaction.

A glucose sensor is an example of (1). In the sensor, glucose oxidase as an enzyme oxidizes β-D-glucose to D-glucono δ-lactone selectively, and oxygen is reduced to hydrogen peroxide. By measuring the amount of oxygen consumed thereupon with an oxygen electrode, it is possible to measure glucose. Methods for measuring the amount of hydrogen peroxide generated thereupon by hydrogen peroxide electrodes using platinum electrodes are also known. Further, methods to use metal complexes such as potassium ferricyanide, ferrocene derivative and quinone derivative as an electron receptor are proposed. Furthermore, types of sensors to use an organic compound as an electron receptor are proposed.

This type of a sensor oxidizes a reductant of electron acceptor provided as a result of enzyme reaction on electrodes to find the glucose level from the oxidation current. Therefore, this type of sensor is broadly applied to measure other substrates not only to glucose. For example, a biosensor is disclosed: forming a system of electrodes comprising measurement electrode, counter electrode and reference electrode on an insulating motherboard using methods of screen printing; and forming a reaction reagent layer including a hydrophilic polymer, an oxidation-reduction enzyme and an electron acceptor, over this system of electrodes (see Patent Document 1). By adding sample liquid including substrates to the reaction reagent layer, this sensor allows the enzyme to react with the substrates and allows the electron acceptor to be reduced. After the enzyme reaction is completed, the reduced electron receptor is oxidized electrochemically. A substrate level in the sample liquid is found from the resulting oxidation current value.

The method of (2) includes making the latex particles to carry an antibody react with an antigen in plasma to measure an antigen from a degree of aggregation of latex particles. To be more specific, by measuring changes in light transmittance accompanying aggregation of latex particles and changes in the number of suspended particles and variations in the distribution of particle sizes by the aggregation, a specific component in the sample liquid is measured. There is a report of a method to use electric fields to accelerate aggregation of latex particles (see Patent Document 2).

However, with the above-described sensor, when a number of reagents is used, mixing reagents in advance may damage storage stability. To improve this, a cholesterol sensor is disclosed that places a reagent including enzymes and surfactants apart from a reagent including the electron acceptor (see Patent Document 3).

Meanwhile, to make a small amount of sample liquid to measure react in a reaction space provided in a microchip, in the above-described sensor, it is important to control the transfer of sample liquid (hereinafter referred to as “liquid transfer”), because the sample liquid cannot react with a reagent sufficiently if liquid transfer is difficult to control. An example is known in which capillary action is used in liquid transfer control. For example, in an immunochromatographic method (see Patent Document 4), sample liquid is transferred by utilizing capillary action, using a porous absorbent material as a chromatographic medium. Immunochromatograph refers to an analysis method of having sample liquid pass a chromatographic medium carrier in which an antibody is fixed, to bind target substances and the antibody.

Further, Patent Document 5 proposes a method of controlling the transfer and stop of sample liquid by centrifugal force or by combining centrifugal force and capillary action, in a method for high-throughput sequencing of nucleic acids.

Further, a method is proposed that provides electrodes under a flow path and applies voltage, to reduce the surface tension of sample liquid against the surfaces of electrodes, to transfer sample liquid. This method is regarded as a method to use electrowetting phenomenon. For example, a mechanism is disclosed that allows droplets to move dimensionally and arbitrarily, using fluid microstructures having electrodes arranged two-dimensionally, in chemical assays (see Patent Document 6).

Patent Document 1: Japanese Patent Application Laid-Open No. HEI2-62952
Patent Document 2: Japanese Patent Publication No. HEI5-88787

### Patent Document 3: Japanese Patent Application Laid-Open No. 2000-39416

### Patent Document 4: Japanese Patent Application Laid-Open No. 2006-189317

### Patent Document 5: Japanese Translation of a PCT Application Laid-Open No. 2002-534096

### Patent Document 6: Japanese Translation of a PCT Application Laid-Open No. 2005-510347

### DISCLOSURE OF INVENTION

### Problems to be Solved by the Invention

To apply to POCT a method of allowing target substances in sample liquid to mix and react with a reagent and measuring chemical changes and physical changes in the resulting sample liquid, it is preferable to carry out reaction and detection in a disposable chip.

This analysis chip needs to reduce measurement errors to conduct analysis correctly. To reduce measurement errors, the sample liquid needs to be uniform, upon detecting changes having occurred in the sample liquid. Particularly, as disclosed in Patent Document 2, when changes in sample liquid are measured with an optical means such as in absorbance measurement, it is necessary to make the concentration of reagents in the sample liquid more uniform. This is because, in an optical means, light is radiated to part of the sample liquid to measure the absorbance, and therefore measurement errors are likely to occur particularly when the sample liquid is not uniform.

To solve this problem, it is effective to divide, on a chip, between an area for mixing the sample liquid and the regent (reaction area) and an area for detecting changes in the sample liquid (detection area), because, by dividing the detection area and reaction area, it is possible to remove factors of measurement errors that can occur in the reaction area. For example, when there is an excess of a reagent, part of the reagent does not dissolve in the sample liquid, to remain in the sample liquid. If the changes in the sample liquid are detected as is in the area, the changes are detected that the sample liquid is in an ununiform state, and therefore errors are likely to occur. Further, when the reaction area and the detection area are divided, liquid flow forms when the sample liquid is transferred from the reaction area to the detection area, and therefore, stirring effect is obtained. As a result, there is an effect of making the sample liquid uniform further.

In POCT, there are cases where several reagents that react with the sample liquid are needed. At this time, from the viewpoint of storage stability, several reagents are needed to place other positions separately. For example, Patent Document 3 discloses a cholesterol sensor that places a reagent including enzymes and surfactants, apart from a reagent including the electron acceptor. However, the sensor in which reagents are placed separately has a problem of taking a long time to mix reagent components when sample liquid is introduced and mixed with several reagents at once. If sample liquid is introduced where reagent A and reagent B are placed separately on a chip, the concentration of reagent A in the sample liquid increases near the place where A is placed. Meanwhile, the concentration of reagent B in the sample liquid increases near the place where B is placed. A distribution of concentrations is produced in the sample liquid as such, and therefore, the sample liquid is less likely to be uniform on the whole. To improve this, a structure may be employed in which sample liquid is individually supplied to reagents provided apart.

As described above, for an analysis chip applicable to POCT, it is effective to place the reaction area and the detection area apart. Further, it is effective to provide several reaction areas as necessary and place them apart. However, this proposal has not been found till now.

Meanwhile, to obtain a chip in which a detection area and (several) reaction area(s) are placed apart, control of the transfer of sample liquid (liquid transfer) is needed. The method of controlling liquid transfer includes using capillary action in a porous absorbent material as in Patent Document 4. However, with this method, the control to stop the liquid transfer once and resume again is complex. For example, controlling sample liquid to stop in the reaction area during the period it takes to react with a regent and then transfer to the detection area is not possible.

As in Patent Document 5, there is a method to control liquid transfer by centrifugal force or by combining centrifugal force and capillary action. This method, however, requires a mechanism to produce rotation to obtain centrifugal force, and makes the apparatus complicated. Further, it is necessary to design an analysis measurement chip having a strength to withstand centrifugal force, so that shapes and materials of the chip are limited.

As in Patent Document 6, when an electrowetting phenomenon is used to control liquid transfer, a problem occurs in cases where the surface tension of sample fluid against surfaces of electrodes is sufficiently low before voltage is applied, because, by introducing a sample fluid to a capillary voluntarily, the control of liquid transfer is difficult. Particularly, when the surface tension of the sample fluid varies distinctly by the reactions with reagents, for example, when a surfactant is contained in the reagent, the control is especially difficult.

It is therefore an object of the present invention to provide a sample liquid analysis chip that has a reaction area and a detection area apart from each other in a simple structure, and that is capable of controlling the transfer of sample liquid.

### Means for Solving the Problem

The present inventors have found to use a “capillary valve” that controls capillary action by forming discontinuous planes on capillary walls, for the control of the transfer of sample liquid, to make the present invention. That is, the above problem is solved by the following sample liquid analysis chip.

[1] A sample liquid analysis chip including a tubular, open-ended sample liquid flow path, and a reaction reagent layer and a detection area for detecting chemical or physical changes in a sample liquid, the reaction reagent layer and the detection area being placed in an interior wall of the sample liquid flow path, the chip adopts the configuration including: an opening that is provided in part of a wall of the sample liquid flow path; and a lid that fits in the opening to form the part of the wall, wherein a material of the lid forming the part of the wall is the same as a material of the wall near the opening.
[2] The sample liquid analysis chip according to [1], wherein part of the lid and the wall of the sample liquid flow path form a hinge of the sample liquid flow path.
[3] The sample liquid analysis chip according to [1], wherein the lid is separate from the wall of the sample liquid flow path.
[4] The sample liquid analysis chip according to claim [2], wherein part of the lid forming the part of the wall is disposable.
[5] The sample liquid analysis chip according to any of [1] to [4], wherein: the sample liquid is transferred in the sample solution flow path by capillary action; and the transfer of the sample liquid can be stopped by making an interior wall in the sample liquid flow path a discontinuous structure by opening the lid.
[6] The sample liquid analysis chip according to any of [1] to [5], wherein the reaction reagent layer, the opening and the detection area are placed in the sample liquid flow path in order from upstream in transfer direction of the sample liquid.
[7] The sample liquid analysis chip according to any of [1] to [6], wherein the reaction reagent layers are provided in two positions or more in the interior wall of the sample liquid flow path, and the opening is placed between the reaction reagent layers.
[8] The sample liquid analysis chip according to any of [1] to [7], further comprising a sense area that is sensitive to an arrival of the sample liquid or to changes in the reaction reagent layers in the same position or near position where the reaction reagent layer is placed.
[9] The sample liquid analysis chip according to [2] or [3], wherein: the lid has a square or circular shape; and one side of the square or segment of the circular shape of the lid and the wall form a hinge.
[10] The sample liquid analysis chip according to [2], [3] or [9], wherein the hinge is placed orthogonal to the transfer direction in the sample liquid flow path and downstream of the part of the opening opposing the hinge.
[11] The sample liquid analysis chip according to [10], wherein: a notch in a shape of a letter V is formed in the part of the opening opposing the hinge; and a projection in the shape of the letter V fitting in the notch was formed in the lid fitting in the opening.
[12] The sample liquid analysis chip according to any of [2], [3], and [9] to [11] wherein the hinge is placed orthogonal to the transfer direction in the sample liquid flow path and upstream of the part of the opening opposing the hinge.
[13] The sample liquid analysis chip according to any of [1] to [12], wherein an electrode pair is placed in the detection area, the electrode pair configured with two or more divided conductors and having an connecting part with outside.
[14] The sample liquid analysis chip according to any of [1] to [13], wherein a wall of the sample liquid flow path allows electromagnetic waves to pass.
[15] The sample liquid analysis chip according to any of [1] to [14], wherein the reaction reagent layer includes an enzyme that catalyzes a chemical reaction of a specific component in the sample liquid.
[16] The sample liquid analysis chip according to any of [1] to [15], wherein the reaction reagent layer includes substances that bind selectively with a specific component in the sample liquid.
[17] The sample liquid analysis chip according to [16], wherein the substances that bind selectively with a specific component in the sample liquid comprises an antibody.

Further, the above problem is solved by manufacturing methods of the following sample liquid analysis chip.

[18] A method of manufacturing the sample liquid analysis chip according to [2], includes the steps of: (a) forming a reaction reagent layer on a film motherboard; (b) providing an opening and a lid fitting in the opening on a film ceiling plate; and (c) forming an open ended sample liquid flow path by fixing the motherboard and the ceiling plate at both ends of the motherboard via spacers.
[19] The method of manufacturing the sample liquid analysis chip according to [18], wherein: part of the lid in step (b) and the wall of the sample liquid flow path form a hinge; and the step of providing the lid comprises cutting the wall of the sample liquid flow path leaving part forming the hinge.

Further, the above problem is solved by an analysis method using the following sample liquid analysis chip.

[20] An analysis method using the sample liquid analysis chip according [6], includes the steps of: A) introducing the sample liquid in an analysis chip while the opening that is placed the downstream of and adjacent to the reaction reagent layer with respect to the transfer direction of sample liquid is open to allow the sample liquid to react with a reaction reagent; B) transferring the sample solution to a detection area by closing the opening with the lid after the reaction is finished; and C) detecting chemical changes or physical changes in the sample solution in the detection area.
[21] The analysis method according to [20], wherein the detection step of step (C) comprises detection by an optical technique.
[22] The analysis method according to [22], wherein the detection step of step (C) comprises detection by an electric technique.

### ADVANTAGEOUS EFFECTS OF INVENTION

The present invention provides a sample liquid analysis chip and analysis method thereof that has a reaction area and a detection area apart from each other in a simple structure, that is capable of controlling the transfer of sample liquid, and that is excellent in accurate and quick measurement.

### BRIEF DESCRIPTION OF DRAWINGS

FIG. 1 is a perspective view showing the first aspect of the sample liquid analysis chip;

FIG. 2 shows the structures of a lid and a hinge provided in a flow path of sample liquid;

FIG. 3 is an exploded perspective view showing a second aspect of the sample liquid analysis chip;

FIG. 4 is a cross-sectional view of the sample liquid analysis chip shown in FIG. 3 cutting along the sample liquid flow path;

FIG. 5 is an exploded perspective view showing the sample liquid analysis chip having electrodes;

FIG. 6 is a perspective view showing a third aspect of the sample liquid analysis chip;

FIG. 7 is a perspective view showing a fourth aspect of the sample liquid analysis chip;

FIG. 8 shows an example of the lids of the sample liquid analysis chip; and

FIG. 9 illustrates an analysis method using the sample liquid analysis chip.

### BEST MODE FOR CARRYING OUT THE INVENTION

The sample liquid analysis chip of the present invention has a sample liquid flow path, a reaction area (reaction reagent layer) and a detection area, wherein an opening part is formed in the walls of the sample liquid flow path, and the chip further has a lid that fits in the opening to form part of the walls. The material of the lid configures part of the walls of the sample liquid flow path, and therefore is preferably the same as the material configuring the walls (particularly, the material near part of the opening). The lid fitting in the opening may be unified with the sample liquid analysis chip itself, or may be provided separately. Hereinafter, each aspect will be explained.

1-1. An Aspect in which the Sample Liquid Analysis Chip Itself and the Lid are Unified

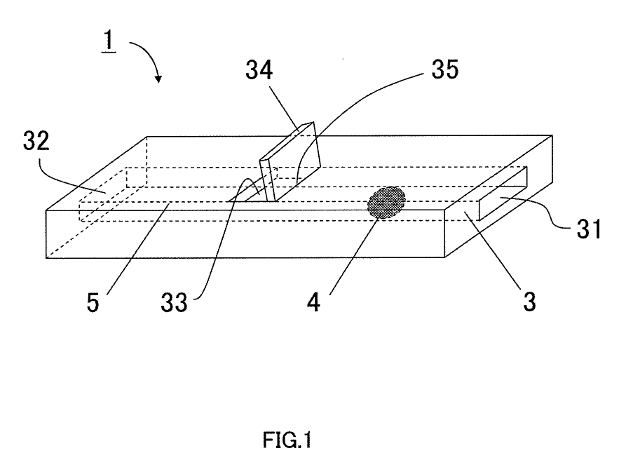

FIG. 1 shows an example of one aspect of the sample liquid analysis chip of the present invention. Referring to FIG. 1, reference numeral 1 denotes the sample liquid analysis chip, which has sample liquid flow path 3, reaction reagent layer 4 and detection area 5. Reference numeral 33 denotes an opening, and reference numeral 34 denotes a lid fitting in the opening. Part of the lid and walls of the sample liquid flow path form hinge 35. Reference numeral 31 denotes a sample liquid inlet and reference numeral 32 denotes an air hole. Hereinafter, with reference to the drawings, components will be explained by assigning reference numerals in figures as necessary.

(1) Liquid Transfer Control

Sample liquid analysis chip 1 of the present invention has tubular, open-ended sample liquid flow path 3. A sample liquid analysis chip refers to a very small member for using analyzing target sample liquid. Tubular, open-ended sample liquid flow path 3 is the flow path for transporting sample liquid, that has openings functioning as sample liquid inlet 31 and air hole 32 at both ends.

For the method of transferring sample liquid to sample liquid flow path 3, a method of using capillary action resulting from the wettability of sample liquid with respect to the walls of the sample liquid flow path may be used. Further, a method of applying pressure to the sample liquid from the outside may be used. In particular, the method of transferring sample liquid to sample liquid flow path 3 is preferable using capillary action because it provides a simple structure without an apparatus that applies pressure.

When a capillary is inserted in liquid vertically, the height of capillary rise by the force the liquid tries to intrude in the capillary, has relationships as the following equation (Tajima, K. et al., “Kaimen Kagaku (Interface Chemistry)”, Maruzen, 2005, p. 41.)

rhρg=2γ cos θ

h: height of capillary rise

r: capillary radius

ρ: liquid density

g: gravitational acceleration

γ: surface tension of liquid

θ: contact angle between capillary surface and liquid

From the above equation, when the contact angle between the capillary surface and liquid is equal to or more than 90 degrees, the right hand side becomes zero or a negative value, and the height of capillary rise h also becomes zero or a negative value. That is, liquid cannot intrude in the capillary. Therefore, it is found that capillary rise does not occur unless the contact angle between the capillary surface and liquid is 90 degrees or less. That is, when sample liquid is transferred by capillary action, the material of walls of the flow path needs to be sufficiently wet with respect to the sample liquid or with respect to liquid in which the reagents in the reaction reagent layer are dissolved. When surfaces of interior walls with respect to the sample liquid flow path are less wet, to improve the wettability of the surfaces of interior walls, it is preferable to treat the surfaces with a surfactant, for example.

Further, the dimensions of the tube forming the sample liquid flow path have influence on transfer of sample liquid by capillary action. In the present invention, in cases where the tube provides a square cross section, the cross section of the tube preferably has a width of 0.5 to 5 mm and a height of 5 to 500 μM. The material forming the tube of the sample liquid flow path is not especially limited, and preferably a polymer film including PET (polyethylene terephthalate) film and polycarbonate film. Further, the above material is preferably treated with a surfactant. Surfactants that are known in the art are used. Examples of surfactants include TritonX-100 (polyoxyethylene-p-t-octylphenyl ether) and butanol solution of phosphatidylcholine. Methods of surfactant treatment known in the art may be used, for example, a surfactant may preferably coat the above material and may be dried. Surfactant treatment may coat all the interior walls forming the flow path, or may coat part of the interior walls, for example, only the ceiling surface in cases where the cross-section is a tubular square. Further, a commercially available PET film subjected to surface treatment in advance may be used.

When the method of applying pressure to the sample liquid from the outside is used, the material of the walls of the sample liquid flow path may not be sufficiently wet with respect to the sample liquid or with respect to liquid in which reagents in a reaction reagent layer are dissolved, because applying pressure from the outside allows the sample liquid to intrude forcibly in the sample liquid flow path. Examples of means for applying pressure to the sample liquid from the outside include a pump or centrifugal force by rotation. Further, in this case, it is preferable to form the flow path using the above-described polymer film. Furthermore, this case has an advantage that the width and height of the flow path are not limited.

The sample liquid analysis chip of the present invention has opening 33 and lid 34 fitting in the opening on part of walls of sample liquid flow path 3, and part of the lid and the walls form hinge 35. The opening refers to a hole provided on the walls of the sample liquid flow path and penetrating through the outside. The opening has a role of providing discontinuous parts in the sample liquid flow path to stop liquid transfer. The lid fitting in the opening is a door provided such that the opening is almost completely covered when the lid is closed. The lid has a role of resolving the discontinuous structure in the sample liquid flow path, to provide a continuous structure. The hinge refers to the part connecting the lid with the walls of the sample liquid flow path.

Assuming that there are opening parts or spatially discontinuous parts due to a step in a capillary, sample liquid intruding in the capillary by capillary action stops moving at the discontinuous parts. This is referred to as “capillary valve phenomenon.” The sample liquid analysis chip of the present invention opens the opening part provided on part of the walls of the flow path by operations from the outside to produce discontinuous parts in the flow path. As a result, it is possible to stop the sample liquid by the above capillary valve phenomenon. Further, by closing the opening, the discontinuous parts of the flow path is resolved, and therefore, it is possible to transfer the sample liquid again.

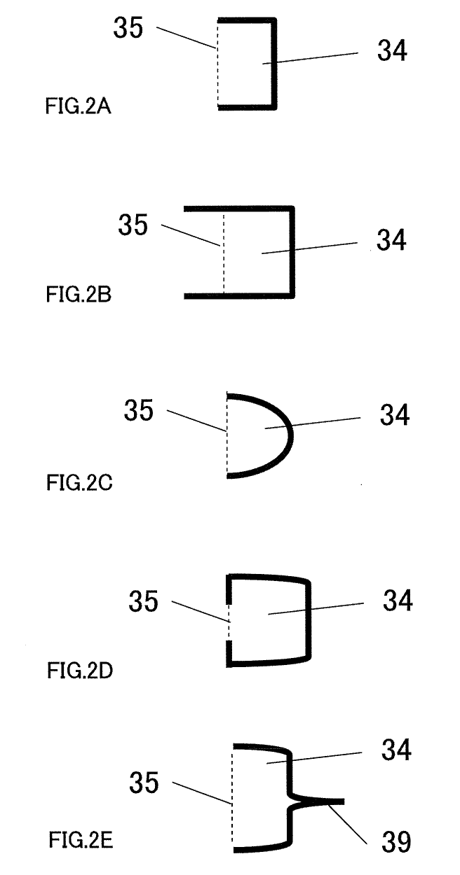

These opening 33 and lid 34 fitting in the opening are provided by cutting part of the walls of the flow path leaving the part to be hinge 35. That is, “the part to be hinge 35” is the part connecting the lid and the walls. The part becomes the hinge, and the part to be the lid is bent outward from the flow path using the hinge as the axis. A lid that functions as a door is formed in this way. The shape of the opening is not especially limited, and preferably has a square or circle shape as shown in FIG. 2, in which one side of the square or segment of the circle and the lid fitting in the opening form the hinge. That is, the opening and the lid fitting in the opening have a Π-shape or C-shape and the part where the line of the letter Π or C breaks preferably forms the hinge.

FIG. 2 shows examples of opening 33 and lid 34 fitting in the opening. While these are provided on the walls of the sample liquid flowing path, the walls are not shown in the figure. Further, reference numerals are the same as in FIG. 1, and therefore the explanations will be omitted (the same applies below). FIG. 2A shows a square shape in which with one side of the square forms a hinge (Π-shape). FIG. 2B shows a square shape in which the hinge is positioned inside one side of the square. FIG. 2C shows a C-shape as described above. Although FIG. 2D shows a configuration in which one side of a square becomes a hinge, part of the hinge is cut. FIG. 2E shows the configuration in which projection 39 is provided at the tip. The configuration of FIG. 2E will be explained in detail later.

The width of the opening and lid (the maximum length of the lid orthogonal to the transfer direction in the flow path) is preferably 30% or above and less than 50% of the perimeter of the interior walls of the cross-section orthogonal to the transfer direction in the flow path, in order to produce an capillary valve effectively and improve the forming workability of the lid. Further, from the same reason, the width of the opening and lid is preferably between 0.5 and 10 mm, and, more preferably, between 0.5 and 5 mm.

When lid 34 is bent to make hinge 35, it is preferable that the hinge does not break. Further, even when the lid provided in this way opens and closes once or more than once, it is preferable that the hinge does not break. The meaning of “the hinge does not break” includes that the hinge does not crack in the vicinity thereof. For this reason, the material of the member forming the lid and hinge is preferably a thermoplastic resin, which is capable of plastic deformation by external force, and which has adequate strength for bends. Examples of thermoplastic resin include polyethylene terephthalate (PET), polycarbonate, polypropylene, polyethylene, polyacetal, polybutyl terephthalate, polyethylene naphthalate and polyamide such as 66 nylon and 6 nylon. A PET is particularly suitable because it has adequate strength and is readily available. Lid 34 and hinge 35 are preferably provided by cutting part of the walls of the sample liquid flow path as described later. In this case, the lid and the hinge are formed from the walls of the sample liquid flow path. Consequently, the lid, the hinge and the sample liquid flow path are made from the same material, and therefore, the above-described materials are preferably used for the material of the sample liquid flow path.

The member to form the lid and hinge preferably has a thickness of 50 to 100 μm, because it allows excellent balance of strength, workability, bending resistance, and suitability for open and close action.

The opening and lid are provided on the walls of the flow path. The positions of the opening and lid are not especially limited, and are preferably provided on the ceiling part, because the opening and lid are easily formed and the lid opens and closes easily.

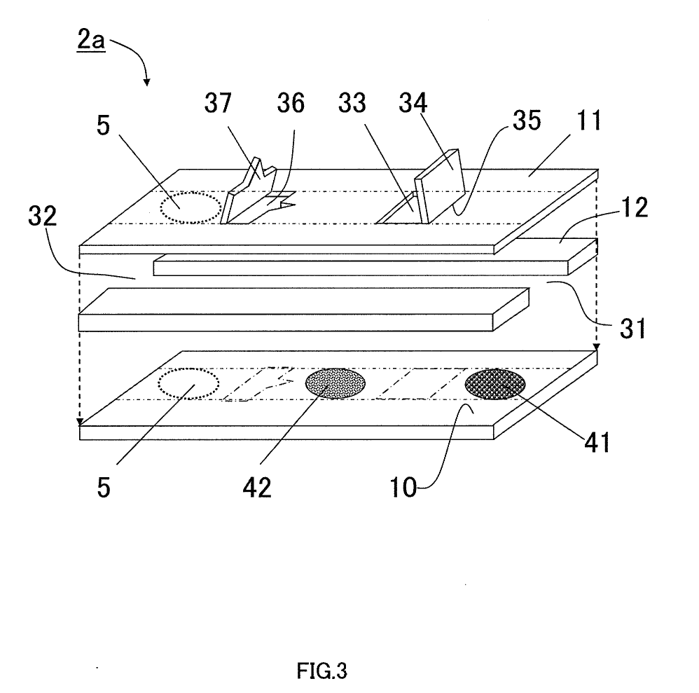

Further, the opening and lid are preferably placed such that the hinge is placed orthogonal to the transfer direction in the sample liquid flow path, and downstream of the part of the opening that opposes the hinge (see lid 37 in FIG. 3). If the opening and lid are provided in this structure and sample liquid is introduced from upstream of the flow path in the situation while the lid is open, the flow path is sure to be divided into part where the sample solution is present and part where the sample solution is not present. If the lid is not open enough, it is difficult for the sample liquid to contact the lid. Therefore, the capillary valve effect makes it possible to stop the sample liquid surely. Next, when the opening is closed with the lid, the discontinuous structure on the interior walls is resolved, and therefore, the sample liquid is transferred by capillary action again. As described above, the accuracy of liquid transfer control improves when the opening and lid are placed such that the hinge is placed orthogonal to the transfer direction in the sample liquid flow path, and downstream of the part of the opening opposing the hinge.

When the lid and opening of above described structure are formed by cutting part of the walls of the flow path, the lid and opening may not adhere completely due to deformation (e.g. bend) during the process. Actually, in most cases, the surface of sample liquid that is stopped at the boundary of the opening bulges slightly toward the downstream of the opening. Consequently, it is generally not a problem even if the lid and the opening do not adhere completely. However, to allow the sample liquid to contact the lid surely, it is preferable to form a V-shaped notch in the part of the opening opposing the hinge and form a gable-shaped projection fitting in the notch in the lid fitting in the opening (see notch 39 in FIG. 2E). By a surface tension of the sample liquid, the sample liquid bulges out slightly at the tip of the letter V in the V-shaped notch part, and therefore, when the lid is closed, the sample liquid that bulges out slightly at the tip of the gable-shaped projection of the lid can contact the lid easily, thereby transferring the liquid surely.

The V-shaped notch is preferably an isosceles triangle having a height between 0.2 and 1.0 mm inclusive, and particularly, between 0.3 and 0.7 mm inclusive, and having an angle of the vertex between 15° and 120° inclusive, and particularly, between 15° and 60° inclusive, because the process for providing a notch is easy and the sample liquid can bulge out adequately. The V-shaped notch may be shaped with a narrower angle in the vertex unless there are technical problems and problems with strength.

Even when there is not such a notch or projection, the sample liquid that is still in the opening can bulges out toward downstream if external force is applied to the sample liquid flow path of the present invention to deform the flow path. It is fundamentally the same as pushing a tube containing liquid to push out the liquid from the tip of the tube.

The opening and lid may be provided such that the hinge is placed orthogonal to the transfer direction in the sample liquid flow path, and upstream of the part of the opening opposing the hinge. In this structure, while the sample liquid stops at the opening, the sample liquid bulges out beyond the hinge to part of the lid. Consequently, even if a gap is made between the lid and the opening due to process, the sample liquid bulges out from the edge parts (particularly, both edges) of the door structure when the lid is closed, and is likely to contact the downstream interior walls. For this reason, it is possible to transfer liquid toward the downstream surely. The sample liquid bulges out to part of the lid, and therefore, to make the sample liquid stop, it is necessary to keep the lid open enough.

Hereinbefore, transfer by the capillary valve effect using an opening and a lid fitting in the opening that are provided on part of walls of a flow path has been explained. In addition to this, liquid transfer can be controlled by the opening and the lid even when sample liquid is transferred by external force. Assuming that external pressure transfers the sample liquid where the lid is open, extra liquid starts to flow from the opening, and therefore it is not possible to transfer the liquid downstream from the opening. Then, when the lid is closed and external pressure is applied, it is possible to transfer the liquid downstream.

(2) Reaction Area

The sample liquid analysis chip of the present invention has reaction reagent layer 4 placed in the interior wall forming the sample liquid flow path. Reaction reagent layer 4 is preferably placed at the bottom face of the sample liquid flow path to allow the reaction regent layer 4 to contact the sample liquid. The reaction reagent layer refers to a solid or semisolid (e.g. gel) member including a reagent to progress reactions of specific components in the sample liquid. Specific components in the sample liquid refer to target substances to be main target of analysis.

Examples of a preferable reagent included in the reaction reagent layer include an enzyme that catalyzes the chemical reactions of specific components in sample liquid and substances that bind with the specific components specifically. Examples of enzymes include cholesterol oxidase, cholesterol dehydrogenase that catalyze oxidization of cholesterol, glucose oxidase that catalyzes oxidization of glucose, lipoprotein lipase that catalyze oxidization of triglyceride (neutral lipid) and glycerol dehydrogenase.

If necessary, the reaction reagent layer may include a surfactant for the purpose of activating enzyme reactions. Examples of surfactants include polyoxyethylene-p-t-octylphenyl ether and cholic acid sodium salt. Further, to make the surface shape of the reagent layer uniform upon drying and accelerate dissolution upon depositing sample liquid in spots, an aqueous solution containing taurine, maltitol and carboxymethyl cellulose may be applied. Further, examples of the reaction regent layer include reagents that are preferable to use with the above enzyme reactions and that allows oxidation-reduction reactions such as potassium ferricyanide.

The substances that bind specifically with specific components means the substances that bind chemically or physically with specific components. Antibodies that have specific binding capacity against antigen are this example. Antibodies that are known in the art are used. Examples of antibodies include an anti-albumin antibody, anti-HCG antibody, anti-IgA antibody, anti-IgM antibody, anti-IgE antibody, anti-IgD antibody, anti-AFP antibody, anti-DNT antibody, anti-prostaglandin antibody, anti-human coagulation factor antibody, anti-CRP antibody, anti-HBs antibody, anti-human growth hormone antibody and anti-steroid hormone antibody.

Further, particles carrying antibodies may be used.

Particles carrying antibodies may be arbitrarily selected, for example, magnetic metal powders, Fe₃O₄ particles, γ-Fe₂O₃ particles, Co-γ-Fe₂O₃ particles, and composite microparticles of polymers such as nylon and polyacrylamide and ferrites. These particles can be prepared by conventional methods. For example, as shown in Patent Document 2, these particles may be prepared using a silane coupling agent, for example.

The reaction reagent layer is preferably placed such that the reaction reagent layer is definitely dissolved, spread, or swells when the reaction reagent layer contacts sample liquid. The reaction reagent layer is formed by adding a reagent solution dropwise that is obtained by dissolving a reagent in an adequate solvent, to walls of the sample liquid flow path, and by allowing the resulting reagent solution to air dry to make solid. Further, the reaction reagent layer may be formed by allowing an added reagent solution dropwise to freeze dry to make solid (freeze drying method). The reaction reagent layer formed by the freeze drying method has substantially the same volume as the volume of the added solution dropwise and has a gap inside. Consequently, the reaction reagent layer is dissolved quickly in sample liquid. Further, when the freeze drying method is used, residual water after dry decreases compared with air dry, and therefore the storage stability in enzymes, antibodies and so on improves.

The reaction reagent layer is formed in a disc shape, having a diameter of preferably 0.2-1.5 mm. The height of the disc is not particularly limited, and preferably 50 μm or less. Further, when the reaction reagent layer has a space inside the layer, sample liquid is not blocked by the reaction reagent layer, and therefore the reaction reagent layer may have a height of 50 μm or more. In the present invention, “-” means that the values at the both ends of “-” are inclusive.

The reaction reagent layer may be provided in multiple positions. At that time, the opening can be preferably provided in each gap between positions where the reaction reagent layers are provided. The positions of the reagent, the opening and the detection area will be described later. FIG. 3 shows a second aspect of the sample liquid analysis chip and is an exploded perspective view of the sample liquid analysis chip. Actually, the members are combined as shown by the dotted arrows at both ends to form a chip. In FIG. 3, reference numeral 2a denotes the sample liquid analysis chip, which is formed from motherboard 10, ceiling plate 11, and spacer 12. Reference numeral 41 denotes the first reaction reagent layer, and reference numeral 42 denotes the second reaction reagent layer. Reference numeral 33 denotes the first opening and reference numeral 36 denotes the second opening having lids 34 and 37 fitting in the openings, respectively. Reference numeral 35 denotes the hinge. The dash dotted straight line shown in ceiling plate 11 and motherboard 10 shows the part to form the sample liquid flow path when the members are combined. The dash dotted line also shows the positions where openings 33 and 34 are projected on motherboard 10.

(3) Detection Area

The sample liquid analysis chip of the present invention includes detection area 5 to detect physical or chemical changes in sample liquid. Physical changes in sample liquid mean changes in properties of the sample liquid. Changes in properties mean changes in color, changes in the state from liquid to solid. Further, chemical changes in sample liquid mean to change specific components chemically in the sample liquid into different substances.

The detection area refers to the position used to detect above described changes in sample liquid. That is, the detection area where the above changes can be detected serves as the position to detect the changes optically in the state of sample liquid. In this case, the detection area needs to allow electromagnetic waves for detection (visible light when a visual check is conducted) to pass. Further, when detection is carried out electrically, the detection area refers to the position on a tip where electrodes are formed. In FIG. 3, the area shown by reference numeral 5 is applied.

First, cases of detecting the above changes optically will be explained. The optical detection refers to identifying the behavior of chemical or physical changes based on the absorbance and transmittance of sample liquid for electromagnetic waves having specific wavelengths or based on the appearance of solid substances in sample liquid included in a fixed area. Examples of measuring instruments include a measuring absorbance instrument, an image processing apparatus and so on. Further, the changes may be observed with the naked eye.

To make optical detection possible, the entire chip is preferably formed with transparent materials. Examples of these materials include a thermoplastic resin, preferably, polyethylene terephthalate and polycarbonate. Particularly, polycarbonate is extremely transparent to visible light.

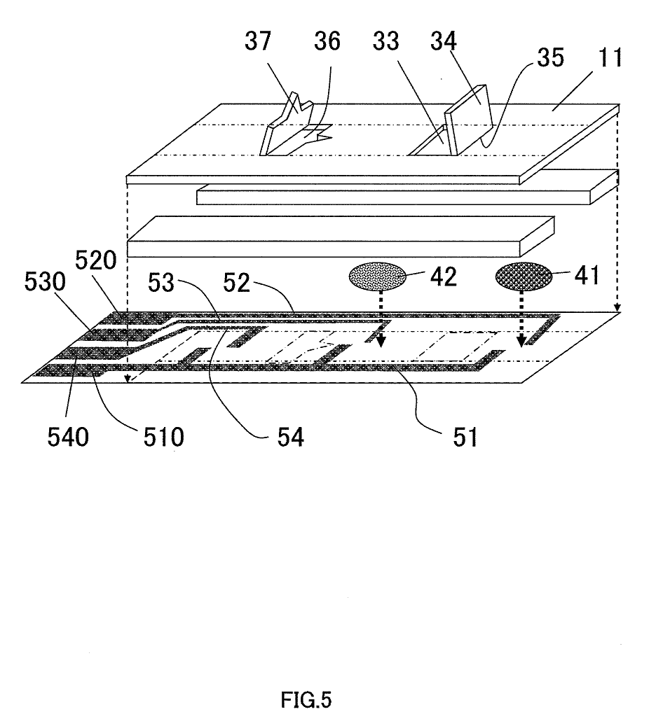

Next, electric detection will be explained. FIG. 5 shows an aspect where electrodes are provided in the detection area in the sample liquid analysis chip. An electrode refers to a conductor or semiconductor provided in pairs to create an electric field or pass a current. In FIG. 5, two divided conductors (reference numerals 51 and 54) form a pair of electrodes. A current value by an oxidation reaction or reduction reaction of substances in the sample liquid or substances generated by chemical reactions between substances in the sample liquid and a reaction reagent is measured using these electrodes. Concentrations of specific substances in the sample liquid are detected from that measured current value. Electrodes formed with two or more divided conductors as such also are referred to as an electrode pair.

An electrode pair is provided in part of the interior walls of the sample liquid flow path, preferably at the bottom of the interior walls. The electrode in the present invention is preferably a planar electrode. A planar electrode is formed, for example, by printing paste containing a conductor, or by using conventional methods such as a sputtering method and deposition method. Further, a planar electrode may be formed by evaporating gold, platinum silver, copper, palladium, chromium, carbon and so on onto a polyethylene terephthalate motherboard surface in thin film layers. More preferably, the conductor materials preferably do not electrochemically react in the sample liquid to ionize, oxidize or reduce.

Further, electrode pairs are preferably connected with an outer port through a lead line, because electrode pairs can be connected with an outer apparatus such as electrochemical measurement apparatuses. Conductors in general may be used for the material of the lead line, preferably, gold, platinum silver and copper. The width of the electrode of the electrode pair is not particularly limited, and, preferably between 0.1 and 1.00 mm inclusive, more preferably, between 0.2 and 0.4 mm inclusive.

(4) Positions of the Reaction Reagent Layer, the Opening and the Detection Area

In the sample liquid analysis chip of the present invention, reaction reagent layer 4, opening 33 and detection area 5 are placed in this order from the upstream in the transfer direction as shown in FIG. 1. The upstream of the transfer direction means closer positions to sample liquid inlet 31 in the sample liquid analysis chip. When reaction reagent layer 4, opening 33 and detection area 5 are placed in this way, the sample liquid introduced and the reaction reagent layer are mixed and react sufficiently in the reaction area. The reaction area is a limited, small space, so that the sample liquid and reaction reagent are mixed and react easily. After this, the sample liquid is further stirred in the process of transferring the reacted sample liquid to the detection area. Consequently, the sample liquid becomes more uniform, and therefore it is possible to detect physical and chemical changes in the sample liquid definitely. That is, by placing reaction reagent layer 4, opening 33 and detection area 5 of the sample liquid analysis chip in this order from the upstream of the transfer direction, it is possible to conduct separately the dissolution of the reaction reagent, chemical reaction between sample liquid and response reagent and detection both in time and space. As a result, it is possible to maintain the uniformity of sample liquid and therefore allow more error-free detection.

For example, in the measurement of an antigen, whereby an antigen in blood reacts with carrier particles carrying an antibody against the antigen, to measure a degree of aggregation of particles, antibody carrier particles are placed as reaction area layer 4. In this measurement, a degree of aggregation is detected based on absorbent measurement or the like, and therefore the concentration of reagents in the sample liquid is required to be more uniform. Then, when reaction reagent layer 4, opening 33 and detection area 5 are placed as shown in FIG. 1, plasma as the sample liquid is mixed and reacts with antibody carrier particles sufficiently in the reaction area, and then moves to detection area 5. Then, by measuring a degree of aggregation of particles, it is possible to measure with few errors.

Further, when reaction reagent layers are provided in multiple positions, the positions where the reaction reagent layers are placed are preferably provided to sandwich openings, because reactions between the sample liquid and individual reaction reagents can be carried out without influence of other reaction reagents.

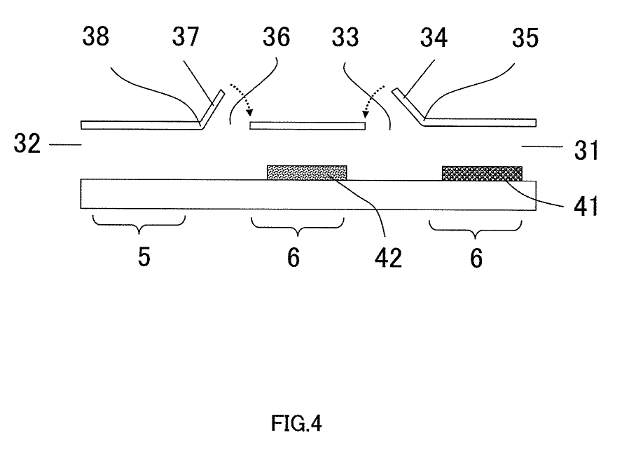

For example, a case of measuring the concentration of cholesterol in plasma will be explained with reference to FIG. 4. A serum cholesterol level used in diagnosis totals the concentrations of cholesterol and cholesterol ester. Generally, cholesterol is measured by the method of oxidizing cholesterol using an enzyme (cholesterol oxidase: ChOD). However, cholesterol ester cannot act as the substrate for oxidation reaction by ChOD, and therefore, the method of using cholesterol esterase (ChE) that changes cholesterol ester into cholesterol is known. That is, the concentration of serum cholesterol is measured by utilizing the following reactions A and B using two kinds of enzymes.

cholesterol ester→cholesterol+fatty acid (reaction by an enzyme ChE) Reaction A

cholesterol+ferrocyanide ion→cholestenone+ferrocyanide ion (reaction by an enzyme ChOD) Reaction B

In this measurement, it is preferable to make a reagent containing ferrocyanide ion as a primary component first reaction reagent layer 41 and a reagent containing ChOD and ChE as primary components second reaction reagent layer 42. First, blood as the sample liquid is mixed with the first reaction reagent. Ferricyanide ion of the first reaction reagent layer cannot react individually with cholesterol or cholesterol ester, and therefore only the reagents are mixed in first reaction reagent layer 41.

Next, the sample liquid is mixed with ChE and ChOD in second reaction reagent layer 42, to change cholesterol ester into cholesterol with the reaction A. Cholesterol (the original cholesterol and the cholesterol generated from reaction A) is oxidized to cholestenone by reaction B, and electrons generated by this reduces ferricyanide ion to ferrocyanide ion. The amount of decrease of ferricyanide ion matches the amount of cholesterol that is decreased by oxidation reaction, and therefore, it is possible to calculate the concentration of cholesterol when the amount of decrease of ferricyanide ion is measured in detection area 5.

If a reagent layer in which all reagents used in these reactions are placed in one position, the reagents deteriorate due to long term storage. To avoid this, it is preferable to place reagents separately on the sample liquid analysis chip as described above.

Further, by providing reagent layers separately, the order of reactions can be controlled to improve the accuracy of measurement. In the above example, if the reagent containing ChOD and ChE as primary components is placed in the first reaction reagent layer (upstream), as soon as sample liquid is mixed with the reaction reagent, reaction B starts, because reaction B advances if there is oxygen in the sample liquid even though there is not ferricyanide ion. When reaction B starts, oxygen is reduced to generate hydrogen peroxide. After that, although ferricyanide ion of the second reaction reagent layer reacts with electrons generated by reaction B, the oxidation reaction has already started, and therefore the concentration of cholesterol cannot be measured accurately if the ferricyanide ion is measured.

As described above, by providing a reaction reagent layer containing potassium ferricyanide as a primary component and a reaction reagent layer containing ChOD and ChE as primary components separately, and by placing the former in the upstream from the latter, the accuracy of measurement improves.

(5) Sense Area

The sample liquid analysis chip of the present invention may have a sense area that is able to sense arrival of sample liquid or changes in the reaction reagent layer in the same position or near the position where the reaction reagent layer is placed. By providing a sense area, it is possible to carry out more accurate analysis. The arrival of sample liquid refers that sample liquid is transferred to the reaction reagent layer. Changes in the reaction reagent layer refers to dissolution and reaction of the reaction reagent layer by mixture with sample liquid. That is, the sense area refers to the position to sense optically or electrically the arrival of sample liquid or changes in the reaction reagent layer. The sense area may be configured by a member that is capable of transmitting electromagnetic waves as in the detection area.

The sense area is preferably placed in the same position or near the position in which the reaction reagent layer is placed, and, more preferably, slightly downstream of the reaction reagent layer, because the above behavior in the reaction regent layer can be observed certainly. To be more specific, the sense area is preferably provided in the positions showing reference numeral 6 in FIG. 4. FIG. 4 is a cross-sectional view cutting sample liquid analysis chip 2a in FIG. 3 along the line linking 31 and 32. Sense areas 6 are placed in the same positions in which first reaction reagent layer 41 and second reaction reagent layer 42, or placed in positions slightly downstream of those positions.

Further, electrodes may be provided in the sense areas as shown in FIG. 5. The parts in which sense areas 51 and 52 are present form an electrode pair, which electrically senses the arrival of sample liquid and changes in the reaction reagent layer. The electrodes in sense areas 51 and 53 function in the same manner. The electrodes may be provided in the same method as in the detection area.

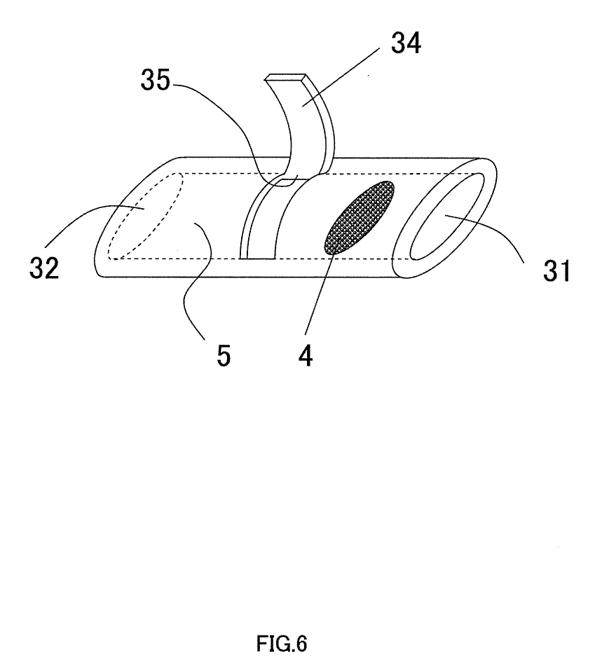

The sample liquid analysis chip of the present invention has been explained above as an example of the sample liquid analysis chip shown in FIG. 1, and an analysis chip as shown in FIG. 6 may be applied. FIG. 6 shows a third aspect of the sample liquid analysis chip. In the sample liquid analysis chip shown in FIG. 6, hinge 35 of the opening and lid 34 fitting in the opening is provided parallel to the transfer direction of sample liquid.

The sample liquid analysis chip of the present invention may have a deposition part to let sample liquid flow easily in the upstream of sample liquid inlet 31. Further, when the sample liquid is blood, solid components including blood cells may be required to be removed, and therefore a filter for removing blood cells may be placed at a given position in a sample liquid flow path.

1-2. The Sample Liquid Analysis Chip Itself and the Lid are Separate

The sample liquid analysis chip of this aspect differs from the above-described chip only in (1) the liquid transfer control, and has the same (2) reaction area (3) detection area and (4) positions of the reaction reagent layer, the opening and the detection area, as the above-described chip. Further, for (1) the liquid transfer control, the sample liquid analysis chip is the same as the above-described chip except that the member of the lid fitting in the opening is a different member from the sample liquid analysis chip itself. Hereinafter, the lid fitting in the opening will be explained mainly.

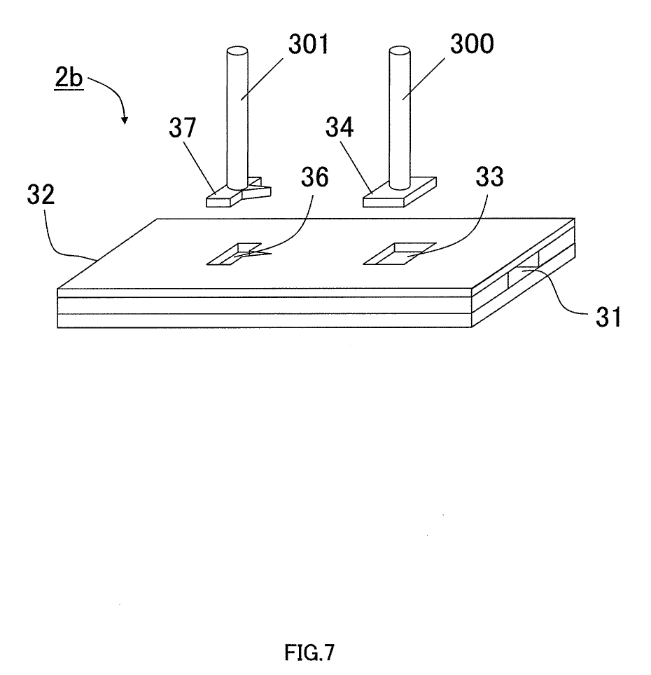

FIG. 7 shows an example of the present aspect of the sample liquid analysis chip. Position relationships between the reagents, the flow paths and so on are the same as in FIG. 3, but differ in that lids 34 and 37 are separated from sample liquid analysis chip 2b. Reference numerals 300 and 301 in the figure serve as retainer tools for carrying lids 34 and 37. These retainer tools 300 and 301 move up, down, left and right to fit lids 34 and 37 retained in the lower end in openings 33 and 36. As the methods of retaining a lid by a retainer tool, for example, by applying negative pressure inside a hollow structured retainer tool, a lid may be held by suction, and, the retainer may be an electromagnet, when a paramagnetic material is used in part of the lid, which is other than the part configuring the inner surface of flow path part in the lid. As the methods of making specific part of a lid a paramagnetic material, a material including paramagnetic particles may be applied, and adhesive tapes made from paramagnetic material may be plastered.

A retainer tool may be incorporated in a measurement apparatus in which the chip is mounted. Although FIG. 7 shows an example in which the measurement apparatus provides the same number (two) of retainer tools as the number of openings, the number of retainer tools is not limited to this. For example, the measurement apparatus may fit lids in openings by moving one retainer tool.

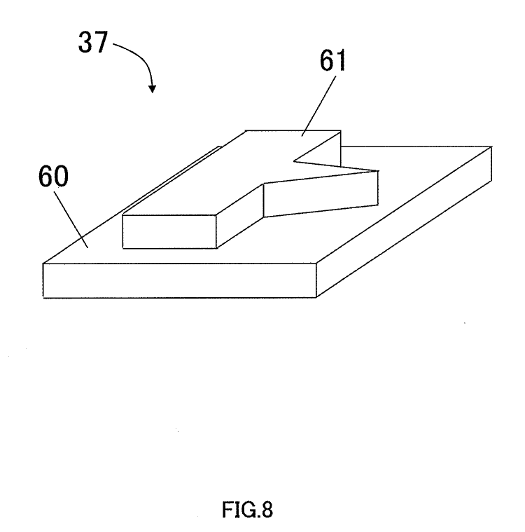

Further, when lids are separate from the sample liquid analysis chip, to improve the closure of the openings and improve the smoothness between the inner surfaces of flow path and the inner surfaces of lids, as shown in FIG. 8, fitting part 61 having the same thickness as ceiling plate 11 may be provided on flat part 60 (the flow path side). This fitting part 61 functions as part of inner surfaces of the flow path when fitting part 61 fits in the opening. This shape makes it possible to resume the transfer of the sample liquid surely when the opening is closed. When these lids are used, the parts forming part of walls (fitting parts 61) may be disposable.

As described above, the sample liquid analysis chip of the present invention has an opening on part of walls in a sample liquid flow path and has a lid fitting in the opening to form the part of the walls, and therefore controls the transfer of sample liquid utilizing capillary valve phenomenon, for example.

2. The Method of Manufacturing a Sample Liquid Analysis Chip of the Present Invention

The sample liquid analysis chip of the present invention may be manufactured by any method as long as the effect of the present invention is not diminished. Hereinafter, preferable manufacturing methods will be explained. The sample liquid analysis chip of the present invention can be manufactured in the following steps: a) forming a reaction reagent layer on a film motherboard; b) providing an opening and a lid fitting in the opening on a film ceiling plate; c) forming an open-ended sample liquid flow path by fixing the motherboard and the ceiling plate at both ends of the motherboard via spacers. This order is not particularly limited, but (c) is preferably the final step from the viewpoint of workability.

As described before, step (a) of forming a reaction reagent layer on a film motherboard, is carried out by adding a reagent solution that is obtained by making a reagent dissolve in an adequate solvent, dropwise to the motherboard, and by allowing the resulting reagent solution to air dry to make solid. It is preferable to use a pipette or dispenser upon adding the same reagent solution dropwise. When using these, the amount of one drop is preferably approximately 50 nl to 5 μl. Particularly, when 50 nl to 1 μl of the solution is added dropwise, it is preferable to use a dispenser. Further, the reagent solution added dropwise is dried and the conditions thereupon may be for ten minutes at 50° C., or for fifteen minutes at humidity less than 60%. Further, the reagent solution may be freeze-dried. The reagent solution can be freeze-dried by usual methods, and, for example, freeze-drying treatment may be carried out as follows. The reagent solution is added dropwise to the motherboard and is immediately pre-freezing. Pre-freezing treatment can be performed by immersing the motherboard in liquid nitrogen or the like. Next, pre-freezing motherboard is left at −40° C. under normal atmospheric pressure, and then is left for 15 minutes at −40° C. in a vacuum atmosphere. Further, temperature increases in the motherboard from −40° C. to −10° C. over a period of two hours, held for one hour at −10° C., increased the temperature from −10° C. to 25° C. over a period of two hours and held for two hours at 25° C.

The position in which the reagent is added dropwise is preferably the position to be interior walls in a sample liquid flow path, and more preferably, the position to be the bottom and the interior walls of the sample liquid flow path. Further, the position to be the interior walls in the sample liquid flow path of the film motherboard may be subject to hydrophilization treatment in advance. As described before, hydrophilization treatment can be performed by coating a surfactant over the walls and drying the surfactant.

Step (b) of providing an opening and a lid fitting in the opening on a film ceiling plate, may be carried out by any method. For example, the lid may be provided by bonding a member obtained by cutting out the opening to part thereof with tape. Further, the lid may be formed by leaving a cut excluding the part to be the hinge in the film ceiling plate and by bending the film using the part to be the hinge as the axis. The latter method is preferable for ease of processing. For the latter method of leaving a cut, it is preferable to use a GRAPHTEC cutting plotter, input a plan view of the cut, and cut so as to fit the shape using a cutter of the cutting plotter. Other than this method, mechanical methods using a corroding blade (pinnacle die), Thompson blade and so on may be used, or, the cut may be formed by laser radiation.

To bend the part where the film is cut using the part to be the hinge as the axis, tweezers and so on may be used. Further, a die in which a male punch fitting in the part to be the lid is provided is prepared, the film may be placed on the die to stamp the film. It is preferable to bend the part to a degree the hinge whitens, that is, to bend the part such that the film is deformed plastically. The bent lid is preferably open at a certain angle with respect to the walls of the sample liquid flow path. This angle is not particularly limited, and, preferably 20° to 60° for the ease of closing the lid.

Then, motherboard 10 where the reaction reagent layers are provided in step (a) and ceiling plate 11 where openings 33 and 36 and lids 34 and 37 are provided in step (b) are fixed together via spacers 12. The spacers are preferably placed at the ends of the chip, as shown in the figures, so as to form sample liquid flow path 3. Motherboard 10 and ceiling plate 11 are fixed together by bonding using adhesive or hot stamping. It is preferable to fix the motherboard and the ceiling plate together using adhesive for ease of processing. Adhesive containing an acrylic resin known in the art may be used.

Further, the sample liquid analysis chip shown in FIG. 6 can be manufactured by forming a reaction reagent layer on a film and providing an opening and a lid fitting in the opening in the film, and by bonding the ends of the film to wind the film.

3. The Analysis Method Using the Sample Liquid Analysis Chip of the Present Invention

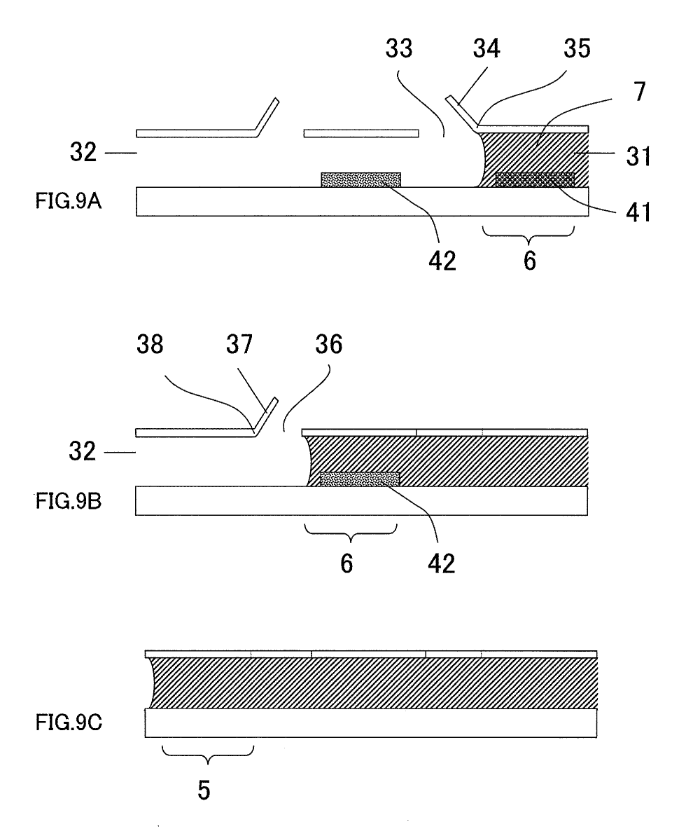

The analysis method will be explained as an example of the sample liquid analysis chip having two reaction reagent layers with reference to FIG. 9. FIG. 9 shows an example of the analysis method using the sample liquid analysis chip, and reference numeral 7 denotes the sample liquid in FIG. 9. First, sample liquid 7 is introduced in the analysis chip from sample liquid inlet 31 while lid 34 that is placed the downstream of and adjacent to first reaction reagent layer 41 with respect to the transfer direction of sample liquid 7 is open (FIG. 9A). Sample liquid 7 is not able to intrude further downstream of opening 33. Sample liquid 7 is mixed with first reaction reagent layer 41 to react in the reaction area formed between sample liquid inlet 31 and the most upstream part (the upstream end) of opening 33. For example, when sample liquid 7 is blood and first reaction reagent layer 41 contains potassium ferricyanide as a primary component, blood and potassium ferricyanide are mixed in this reaction area.

After sample liquid 7 and the reagent in first reaction reagent layer 41 have been mixed sufficiently, opening 33 is closed with lid 34 in the state where opening 36 is open. Sample liquid 7 is transferred up to opening 36, and mixed with second reaction reagent layer 42, to react with the second reaction reagent layer (FIG. 9B). When the second reaction reagent layer contains ChOD and ChE as primary components, ChE changes cholesterol ester in the sample liquid to cholesterol, and ChOD oxidizes the changed cholesterol and original cholesterol to cholestenone. Then, potassium ferricyanide in the sample liquid is reduced to potassium ferrocyanide.

After the reaction has advanced sufficiently, the sample liquid is transferred to detection area 5 when opening 36 is closed, to detect chemical changes or physical changes (FIG. 9C). The detection may be carried out by comparing changes in color visually with color samples prepared in advance. Further, the detection may be carried out by applying pulse voltage to electrodes provided in detection area 5, and then measuring the oxidation current of potassium ferrocyanide to potassium ferricyanide. When potassium ferrocyanide can be measured, cholesterol level in blood can be measured.

The above lids may be pushed directly to close openings using tweezers and so on by the individual conducting the test, and, besides this, may be provided with mechanisms that gives external force. For example, the openings may be closed by pressing a movable bar structure against the lids, or, may be closed by rolling a roller structure having an axis parallel to the hinge, from the hinge to the tip boundary of the door structure.

Although the method of measuring cholesterol in blood has been explained as an example, other measurements may be carried out in the same way. Further, measurements may be carried out in the sense area that is provided near the reaction reagent layers, while checking whether or not the sample liquid is transferred to the first and second reaction reagent layers, and whether or not the sample liquid is mixed with the reagents. For example, when the sense area is formed with a transparent member for specific electromagnetic waves, it is possible to check the sense area visually or with observations of optical changes (changes in transmitting specific electromagnetic waves). Further, changes in potential may be measured by providing electrodes in the place.

### EXAMPLES

### Example 1

### Cholesterol Sensor Shown in FIG. 3

A polycarbonate ceiling plate having a thickness of 0.1 mm, a length of 5 mm and a width of 3 mm was prepared. A plan view of a cuts was inputted to a GRPHTEC cutting plotter, to form the cut in the shapes shown in FIGS. 2A and 2E on the prepared ceiling plate. The formed cut parts were bent as shown in FIG. 3, to form lids (34 and 37) and openings (33 and 36). Hinges (35 and 38) were bent to be whitened so that the lids were left open. The distance from sample liquid inlet 31 to hinge 35 of first opening 33 was 1 mm, and the distance from hinge 38 of second opening 36 to air hole 32 was also 1 mm.

The width of the sample liquid flow path was 0.8 mm, and the width of lids 34 and 37 was 0.8 mm. The hinge of lid 34 was placed upstream of the part opposing the hinge of opening 33. The hinge of lid 37 was placed downstream of the part opposing the hinge of opening 36. A projection of an isosceles triangle having an apex of 30° and a height of 0.3 mm was provided in lid 37, and a notch of the same shape to fit in this was provided in opening 36.

Meanwhile, a polycarbonate motherboard having a thickness of 0.1 mm, a height of 5 mm and a width of 3 mm was prepared. Reaction reagent layers 41 and 42 were formed as follows in the positions on the motherboard where the sample liquid flow path is formed.

<Reaction Reagent Layer 41>

Mixed aqueous solution containing 70 mmol/l of potassium ferricyanide, 0.05 wt % of carboxymethyl cellulose (CMC), 1.33 wt % of taurine, and 0.1 wt % of maltitol was prepared. 0.2 μl of the prepared mixed aqueous solution was added dropwise to a middle position, which was a position of the sample liquid flow path on the motherboard, and which was located between sample liquid inlet 31 and hinge 37 provided in the first opening. The added solution was dried to be made solid in a laboratory at room temperature 25° C. The steps of this dropwise addition and drying were repeated ten times to form a reaction reagent layer. The reagent layer was formed in a disc shape having approximately a 0.8 mm diameter with its center in the middle position between sample liquid inlet 31 and hinge 35, and was accommodated in the sample liquid flow path. Meanwhile, in the case of adding 2.0 μl of the mixed aqueous solution dropwise all at once, the droplet is too spread too much, and the diameter of reagent layer 41 cannot be accommodated within 0.8 mm.

<Reaction Reagent Layer 42>

0.75 kU/ml (about 2.34 wt %) of cholesterol oxidase (ChOD) derived from Nocardia, 2 kU/ml (about 1.1 wt %) of cholesterol esterase (ChE) derived from Pseudomonas, 0.5 wt % of polyoxyethylene-p-t-octylphenyl ether (TritonX-100) as a surfactant having an effect of activating reaction of cholesterol esterase, 75 kU/ml of cholic acid sodium salt as a surfactant, 1 wt % of taurine, 0.25 wt % of maltitol and 0.2 wt % of CMC, were dissolved in water to prepare a mixed solution. Next, 0.2 μl of the mixed solution was added dropwise to a position, where the sample liquid flow path on the motherboard, and which was located between the downstream end of first opening 33 and the upstream end of second opening part 36, and was dried in a room in the same way as reagent layer 41. This operation was performed only once to form reaction reagent layer 42 in a disc shape having a diameter of 0.8 mm. In the present example, reaction reagent layer 41 containing potassium ferricyanide as a primary component was provided the upstream of reaction reagent layer 42 containing ChOD and ChE as primary components.

A PET film having a thickness of 75 μm, a length of 5 mm and a width of 1.1 mm was prepared. An acrylic adhesive having a thickness of 12.5 μm was applied to the upper and bottom surfaces of the PET film to make a spacer (a thickness of 100 μm). By interposing spacers between the motherboard and the ceiling plate to paste these together, a sample liquid analysis chip for cholesterol sensor was formed.

Plasma was supplied as sample liquid to the chip made in this way. 1 μl plasma was added dropwise to a resin strip to form a plasma droplet, and the plasma droplet was made to be contacted with sample liquid inlet 31 of the chip. Plasma was introduced in sample liquid flow path 3. It was confirmed by observing visually at sense area 6 that plasma introduced stopped moving in the position of hinge 35 placed before opening 33. The sample liquid analysis chip had excellent visibility because all members were transparent.

After it was confirmed that reagent layer 41 was dissolved in the plasma, lid 34 was pushed from outside with tweezers, to close opening 33. As a result, the plasma arrived at reaction reagent layer 42, and stopped at the upstream end of opening 36. This was checked by observing sense area 6 visually. Further, it was also checked that plasma bulges a little in the V-shaped notch part of opening 36.

After it was confirmed that reaction reagent layer 42 was dissolved in plasma to blur its outline, opening 36 was closed with lid 37. As a result, the plasma arrived at air hole 32 and stopped. Meanwhile, plasma droplets were kept in contact with sample liquid inlet 31 of the chip, that is, plasma was kept on being supplied from sample liquid inlet 31.

After two minutes had passed since plasma arrived at air hole 32, the color of the plasma present in detection area 5 was observed. At the same time, by comparing the color with color samples in which given concentrations of cholesterol reacted, cholesterol concentration was calculated.

### Example 2

### Cholesterol Sensor Shown in FIG. 5

A motherboard, a ceiling plate, spacers were prepared as shown in FIG. 5 as in Example 1. The ceiling plate was made of PET. Further, four systems of electrodes were formed on motherboard 1 as shown in FIG. 5. These electrodes were made by masking patterns made of stainless steel and by forming a palladium film in a sputtering apparatus. The motherboard had the same width as the ceiling, and had a length of 7.5 mm, which was longer than the ceiling plate. For that reason, when the motherboard and the spacers were pasted together, the motherboard stuck out 2.5 mm from the side of air hole 32.

Terminals (510 to 540) were placed in the area up to 1.5 mm from the downstream end of the part where the motherboard was stuck, to made a connecting part with the outside. Each terminal in the connecting part had a width of 0.5 mm. The interval between each terminal in the connecting part was 0.2 mm. Terminals 510 to 540 were provided in order of 510, 540, 520 and 530 from the front side of the sheet.

A linear conductor (lead line 51) started from terminal 510 placed in the connecting part and ended in the area where reaction reagent layer 41 was provided, was provided on the motherboard. Lead line 51 had: a part that was placed parallel to sample liquid flow path 3 in the area in the front side of the sheet from sample liquid flow path 3 (referred to as “parallel linear part”); a part extending perpendicular to the liquid transfer direction from the upstream end of parallel linear part toward sample liquid flow path 3 (referred to as “perpendicular linear part”); and two parts branching perpendicular to the liquid transfer direction from the parallel linear part toward sample liquid flow path 3 (referred to as the “first branch lead part” and “second branch lead part”).

The parallel linear part had a width of 0.3 mm. Part of the perpendicular linear part extended 0.3 mm into the area in sample liquid flow path 3. Further, the first branch lead part arrived at the position where reaction reagent layer 42 was provided and the second branch lead part arrived at the detection area. Both parts of the first and second lead extended 0.3 mm into the area in sample liquid flow path 3. Each width of the branch lead part was 0.3 mm.

A linear conductor (lead line 52) started from terminal 520 placed in the connecting part and ended in the area where reaction reagent layer 41 was provided, was provided on the motherboard. Lead line 52 had: a part that was placed parallel to sample liquid flow path 3 in the area in the depth side of the sheet from sample liquid flow path 3 (referred to as “parallel linear part”); and a part extending perpendicular to the liquid transfer direction from the upstream end of the parallel linear part toward sample liquid flow path 3 (referred to as “perpendicular linear part”). The lead line had a width of 0.3 mm. Further, part of the perpendicular linear part extended 0.3 mm into the area in sample liquid flow path 3.

A linear conductor (lead line 53) started from terminal 530 placed in the connecting part and ended in the same position as the position where reaction reagent layer 42 was provided, was provided on the motherboard. Lead line 53 had: a part that was placed parallel to sample liquid flow path 3 in the area in the depth side of the sheet from sample liquid flow path 3 and in the front side of the sheet from lead line 52 (referred to as “parallel linear part”); a part extending perpendicular to the liquid transfer direction from the upstream end of the parallel linear part toward sample liquid flow path 3 (referred to as “perpendicular linear part”); and a linear part to connect the downstream end of the parallel linear part and terminal 530. The downstream end of the parallel linear part was located in slightly downstream of the downstream end of ceiling plate 11, in a position not touching terminal 520. The lead line had a width of 0.3 mm. Further, part of the perpendicular linear part extended 0.3 mm into the area in sample liquid flow path 3.

A linear conductor (lead line 54) started from terminal 540 placed in the connecting part and ended in the same position as the position where the detection area was provided, was provided on the motherboard. Lead line 54 had: a part that was placed parallel to the sample liquid flow path 3 in the area in the depth side of the sheet from sample liquid flow path 3 and in the front side of the sheet than lead line 53 (referred to as “parallel linear part”); a part extending perpendicular to the liquid transfer direction from the upstream end of the parallel linear part toward sample liquid flow path 3 (referred to as “perpendicular linear part”); and a linear part to connect the downstream end of the parallel linear part and terminal 540. The downstream end of the parallel linear part was located in slightly downstream of the downstream end of ceiling plate 11, and in a position not touching terminal 530. The lead line has a width of 0.5 mm. Further, part of the perpendicular linear part extended 0.5 mm into the area in sample liquid flow path 3.

The above conductors formed pairs to configure electrode pairs. In the present example, the electrode pairs configured with “terminals 510 and 520” and “terminals 510 and 530” were used to sense whether or not a sample liquid arrived during the transfer of the sample liquid by changes in electrical resistance values. Further, the electrode pair configured with “terminals 510 and 540” was used to measure electrochemical changes in the sample liquid after the sample liquid arrived at the detection area.

Spots of plasma were supplied to the sample liquid analysis chip in the same method as in Example 1. Before the supply, the resistance value between terminal 510 and terminal 520 started to be measured. It was checked that the resistance value decreased suddenly when the plasma arrived at the upstream end of first opening 33. The resistance value between terminal 510 and terminal 520 was immediately finished being measured, and, at the same time, the resistance value between terminal 510 and terminal 530 was started to be measured.

After the decrease of the resistance value between terminal 510 and terminal 520 was confirmed, 10 seconds were spent waiting, to provide a period to allow reaction reagent layer 41 to dissolve and spread. After that, opening 33 was closed with lid 34 in the same manner as in Example 1. After several seconds since opening 33 was closed, a decrease in resistance value between terminal 510 and terminal 530 was observed. The resistance value between terminal 510 and terminal 530 was immediately finished being measured, and, at the same time, the resistance value between terminal 510 and terminal 540 was started to be measured.

After the decrease of the resistance value between terminal 510 and terminal 530 was confirmed, 10 seconds were spent waiting, to provide a period to allow reaction reagent layer 42 to dissolve and spread. After that, opening 36 was closed with lid 37 in the same manner as in Example 1. After several seconds since opening 36 was closed, a decrease in resistance value between terminal 510 and terminal 540 was observed. The resistance value between terminal 510 and terminal 540 was immediately finished being measured. 2 minutes were spent waiting, to provide a period to allow an oxidation reaction of cholesterol to advance. After that, +0.4 pulse voltage was applied to terminal 540 in relation to terminal 510 in the anode direction, and a current value after 3 seconds was measured. The measured current value showed response depending upon total cholesterol concentration.

### Example 3

### Examination of the Position of the Hinges

A cholesterol sensor was made in the same manner as in Example 2. However, the structures of the first opening and the lid were switched to the structures of the second opening. That is, first lid 34 was prepared such that hinge 35 was placed in the downstream of the upstream end of opening 33. A projection of an isosceles triangle having an apex of 30° and a height of 0.3 mm was provided in lid 34, and a notch of the same shape to fit in this was provided in opening 33. Second lid 37 was provided such that the hinge was placed the upstream of the downstream end of opening 36. Lid 37 had a rectangle shape without projections. Opening 36 had a rectangle shape fitting in this lid. A notch having the same shape as the lid was provided.

The sample liquid analysis chip having a lid, which had the same square shape as first lid 34 in Example 2 and whose hinge was placed parallel to the flow path was prepared, and the same experiment was conducted as in Example 2. As a result, it was confirmed that good transfer control is possible.

### Example 4

### Examination of the Shapes of Lids

The sample liquid analysis chip in which first lid 34 in Example 2 was shaped as shown in FIG. 2B, 2C or 2D, was prepared. The lid in a shape of FIG. 2B or 2C was formed in the same manner as in Example 1. The lid in a shape of FIG. 2D was formed by a lid having a width of 0.8 mm as in Example 1, and formed by cutting both edges of the hinge by 0.1 mm. When a measurement test was conducted as in Example 2 using the sample liquid analysis chip prepared in this way, it was confirmed that the same control transfer as in Example 2 was possible.

The lid in FIG. 2C was not slightly different from the lid in FIG. 2A in forming process. The lid in FIG. 2B was formed more easily than the lid in FIG. 2A in bending the hinge. The lid in FIG. 2D was formed easily in bending the hinge, and the step of closing the lid was easy when analysis was conducted.

The disclosure of Japanese Patent Application No. 2007-099693, filed on Apr. 5, 2007, including the specification, drawings and abstract, is incorporated herein by reference in its entirety.

### INDUSTRIAL APPLICABILITY

The sample liquid analysis chip of the present invention is capable of analyzing quickly and definitely therein in a simple structure. For this reason, the sample liquid analysis chip of the present invention is suitable for use in analysis apparatuses for POCT, for example, a blood component measurement apparatus.

### EXPLANATION OF REFERENCE NUMERALS

1, 2A and 2B SAMPLE LIQUID ANALYSIS CHIP
10 MOTHERBOARD
11 CEILING PLATE
12 SPACER
3 SAMPLE LIQUID FLOW PATH
31 SAMPLE LIQUID INLET
32 AIR HOLE
33 and 36 OPENING
34 and 37 LID
35 and 38 HINGE
39 NOTCH
300 and 310 RETAINER TOOL
4 REACTION REAGENT LAYER
41 FIRST REACTION REAGENT LAYER
42 SECOND REACTION REAGENT LAYER
5 DETECTION AREA
51, 52, 53 and 54 CONDUCTOR
510, 520, 530 and 540 TERMINAL
6 SENSE AREA FOR DETECTING INTRODUCE OF SAMPLE LIQUID
60 FLAT PART
61 FITTING PART
7 SAMPLE LIQUID

1. A sample liquid analysis chip including a tubular, open-ended sample liquid flow path, and a reaction reagent layer and a detection area for detecting chemical or physical changes in a sample liquid, the reaction reagent layer and the detection area being placed in an interior wall of the sample liquid flow path, the chip comprising:
an opening that is provided in part of a wall of the sample liquid flow path; and
a lid that fits in the opening to form the part of the wall,
wherein a material of the lid forming the part of the wall is the same as a material of the wall near the opening.

2. The sample liquid analysis chip according to claim 1, wherein part of the lid and the wall of the sample liquid flow path form a hinge of the sample liquid flow path.

3. The sample liquid analysis chip according to claim 1, wherein the lid is separate from the wall of the sample liquid flow path.

4. The sample liquid analysis chip according to claim 3, wherein part of the lid forming the part of the wall is disposable.

5. The sample liquid analysis chip according to claim 1, wherein:
the sample liquid is transferred in the sample solution flow path by capillary action; and
the transfer of the sample liquid can be stopped by making an interior wall in the sample liquid flow path a discontinuous structure by opening the lid.

6. The sample liquid analysis chip according to claim 1, wherein the reaction reagent layer, the opening and the detection area are placed in the sample liquid flow path in order from upstream in transfer direction of the sample liquid.

7. The sample liquid analysis chip according to claim 6, wherein the reaction reagent layers are provided in two positions or more in the interior wall of the sample liquid flow path, and the opening is placed between the reaction reagent layers.

8. The sample liquid analysis chip according to claim 1, further comprising a sense area that is sensitive to an arrival of the sample liquid or to changes in the reaction reagent layers in the same position or near position where the reaction reagent layer is placed.

9. The sample liquid analysis chip according to claim 2, wherein:
the lid has a square or circular shape; and
one side of the square shape or segment of the circular shape of the lid and the wall form a hinge.

10. The sample liquid analysis chip according to claim 2, wherein the hinge is placed orthogonal to the transfer direction in the sample liquid flow path and downstream of the part of the opening opposing the hinge.

11. The sample liquid analysis chip according to claim 10, wherein:
a notch in a shape of a letter V is formed in the part of the opening opposing the hinge; and
a projection in the shape of the letter V fitting in the notch was formed in the lid fitting in the opening.

12. The sample liquid analysis chip according to claim 2, wherein the hinge is placed orthogonal to the transfer direction in the sample liquid flow path and upstream of the part of the opening opposing the hinge.

13. The sample liquid analysis chip according to claim 1, wherein an electrode pair is placed in the detection area, the electrode pair configured with two or more divided conductors and having an connecting part with outside.

14. The sample liquid analysis chip according to claim 1, wherein a wall of the sample liquid flow path allows electromagnetic waves to pass.

15. The sample liquid analysis chip according to claim 1, wherein the reaction reagent layer includes an enzyme that catalyzes a chemical reaction of a specific component in the sample liquid.

16. The sample liquid analysis chip according to claim 1, wherein the reaction reagent layer includes substances that bind selectively with a specific component in the sample liquid.

17. The sample liquid analysis chip according to claim 1, wherein the substances that bind selectively with a specific component in the sample liquid comprises an antibody.

18. A method of manufacturing the sample liquid analysis chip according to claim 2, comprising the steps of:
(a) forming a reaction reagent layer on a film motherboard;
(b) providing an opening and a lid fitting in the opening on a film ceiling plate; and
(c) forming an open ended sample liquid flow path by fixing the motherboard and the ceiling plate at both ends of the motherboard via spacers.

19. The method of manufacturing the sample liquid analysis chip according to claim 18, wherein:
part of the lid in step (b) and the wall of the sample liquid flow path form a hinge; and
the step of providing the lid comprises cutting the wall of the sample liquid flow path leaving part forming the hinge.

20. An analysis method using the sample liquid analysis chip according to claim 6, comprising the steps of:
A) introducing the sample liquid in an analysis chip while the opening that is placed the downstream of and adjacent to the reaction reagent layer with respect to the transfer direction of sample liquid is open to allow the sample liquid to react with a reaction reagent;
B) transferring the sample solution to a detection area by closing the opening with the lid after the reaction is finished; and
C) detecting chemical changes or physical changes in the sample solution in the detection area.

21. The analysis method according to claim 20, wherein the detection step of step (C) comprises detection by an optical technique.

22. The analysis method according to claim 22, wherein the detection step of step (C) comprises detection by an electric technique.
